(19) United States
(12) Patent Application Publication
(10) Pub. No.: US 2012/0296130 A1
(43) Pub. Date: Nov. 22, 2012

(54) METHOD FOR ALKYLATION OF TOLUENE TO FORM STYRENE UTILIZING AN OXY-DEHYDROGENATION REACTOR

(75) Inventor: Joseph E. Pelati, Houston, TX (US)

(73) Assignee: FINA TECHNOLOGY, INC., Houston, TX (US)

(21) Appl. No.: 13/457,505

(22) Filed: Apr. 27, 2012

Related U.S. Application Data
(60) Provisional application No. 61/488,777, filed on May 22, 2011.

(51) Int. Cl. C07C 2/86

(52) U.S. Cl. 585/323

(57) ABSTRACT

A process for making styrene is disclosed that includes reacting toluene with a C₁ source over a catalyst in a first reactor producing a first product stream. The first product stream is separated in one or more separation units, wherein a liquid fraction including styrene and ethylbenzene is fed to a second reactor and a gas fraction including carbon monoxide and hydrogen is fed to a third reactor. The gas fraction is oxidized in the third reactor producing a third product stream including carbon dioxide. The third product stream is fed into the second reactor, wherein styrene and ethylbenzene are produced in a second product stream.

### CROSS-REFERENCE TO RELATED APPLICATIONS

This application claims priority to U.S. Provisional Patent No. 61/488,777 filed on May 22, 2011.

### FIELD

The present invention relates to a method for the production of styrene and ethylbenzene. More specifically, the invention relates to the alkylation of toluene with a carbon source (herein referred to as a C₁ source) such as methanol and/or formaldehyde, to produce styrene and ethylbenzene utilizing an oxy-dehydrogenation reactor.

### BACKGROUND

Styrene is a monomer used in the manufacture of many plastics. Styrene is commonly produced by making ethylbenzene, which is then dehydrogenated to produce styrene. Ethylbenzene is typically formed by one or more aromatic conversion processes involving the alkylation of benzene.

Aromatic conversion processes, which are typically carried out utilizing a molecular sieve type catalyst, are well known in the chemical processing industry. Such aromatic conversion processes include the alkylation of aromatic compounds such as benzene with ethylene to produce alkyl aromatics such as ethylbenzene. Typically an alkylation reactor, which can produce a mixture of monoalkyl and polyalkyl benzenes, will be coupled with a transalkylation reactor for the conversion of polyalkyl benzenes to monoalkyl benzenes. The transalkylation process is operated under conditions to cause disproportionation of the polyalkylated aromatic fraction, which can produce a product having an enhanced ethylbenzene content and reduced polyalkylated content. When both alkylation and transalkylation processes are used, two separate reactors, each with its own catalyst, can be employed for each of the processes.

Ethylene is obtained predominantly from the thermal cracking of hydrocarbons, such as ethane, propane, butane, or naphtha. Ethylene can also be produced and recovered from various refinery processes. Thermal cracking and separation technologies for the production of relatively pure ethylene can account for a significant portion of the total ethylbenzene production costs.

Benzene can be obtained from the hydrodealkylation of toluene that involves heating a mixture of toluene with excess hydrogen to elevated temperatures (for example 500° C. to 600° C.) in the presence of a catalyst. Under these conditions, toluene can undergo dealkylation according to the chemical equation: C₆H₅CH₃+H₂→C₆H₆+CH₄. This reaction requires energy input and as can be seen from the above equation, produces methane as a byproduct, which is typically separated and may be used as heating fuel for the process.

Another known process includes the alkylation of toluene to produce styrene and ethylbenzene. In this alkylation process, various aluminosilicate catalysts can be utilized to react methanol and toluene to produce styrene and ethylbenzene. The aluminosilicate catalysts are typically prepared using solutions of acetone and other highly flammable organic substances, which can be hazardous and require additional drying steps. For instance a typical aluminosilicate catalyst can include various promoters supported on a zeolitic substrate. These catalysts can be prepared by subjecting the zeolite to an ion-exchange in an aqueous solution followed by a promoter metal impregnation using acetone. This method requires an intermediate drying step after the ion-exchange to remove all water prior to the promoter metal impregnation with acetone. After the promoter metal impregnation the catalyst is subjected to a further drying step to remove all acetone. This intermediate drying step typically involves heating to at least 150° C., which results in increased costs.

Additionally, in the process of the alkylation of toluene to produce styrene and ethylbenzene, it is often desirable to achieve a high yield of styrene. One known method of achieving a high yield of styrene includes feeding the ethylbenzene into a conventional dehydrogenation reactor. The ethylbenzene may be dehydrogenated to form styrene. However, a large quantity of energy is required to dehydrogenate the ethylbenzene in a conventional dehydrogenation reactor. For example, a molar ratio of steam:oil can be at least 6:1 and in some instances, as high as 8:1 and 9:1. Such high molar ratios of steam to oil require a large dehydrogenation reactor and substantial capital investment. These requirements may not be feasible for certain production facilities.

In view of the above, it would be desirable to have a process of producing styrene and/or ethylbenzene that does not rely on thermal crackers and expensive separation technologies as a source of ethylene. It would further be desirable to avoid the process of converting toluene to benzene with its inherent expense and loss of a carbon atom to form methane. It would be desirable to produce styrene without the use of benzene and ethylene as feedstreams. It would also be desirable to produce styrene and/or ethylbenzene in one reactor without the need for separate reactors requiring additional separation steps. Furthermore, it is desirable to achieve a process having a high yield and selectivity to styrene and ethylbenzene. Even further, it is desirable to achieve a process having a high yield and selectivity to styrene such that the step of dehydrogenation of ethylbenzene to produce styrene can be reduced. It is also desirable to achieve a lower cost alternative having a smaller footprint for the dehydrogenation of ethylbenzene to produce styrene. It is further desirable to be able to produce a catalyst having the properties desired without involving flammable materials and/or intermediate drying steps.

### SUMMARY

An embodiment of the present invention, either by itself or in combination with other embodiments, is a process for making styrene that includes reacting toluene with a C₁ source in the presence of a catalyst in a first reactor to form a first product stream. The first product stream is separated into a gas fraction comprising carbon monoxide and a liquid fraction comprising ethylbenzene. The gas fraction is then reacted with oxygen to form a co-feed that comprises carbon dioxide that is reacted with at least a portion of the liquid fraction in a second reactor to form a second product stream comprising styrene. The gas fraction can include carbon monoxide. The liquid fraction can include styrene and ethylbenzene. The first reactor can be an alkylation reactor and the second reactor can be an oxy-dehydrogenation reactor.

In an embodiment, either by itself or in combination with other embodiments, the liquid fraction is fed into the oxy-dehydrogenation reactor with the carbon dioxide of the co-feed and is dehydrogenated to form the second product stream comprising styrene. The molar ratio of styrene to ethylbenzene in the second product stream can be greater than the molar ratio of styrene to ethylbenzene in the first product stream.

In an embodiment, either by itself or in combination with other embodiments, the C₁ source can be chosen from methanol, formaldehyde, formalin, trioxane, methylformcel, paraformaldehyde, methylal, dimethyl ether, and combinations thereof.

In an alternate embodiment the catalyst can include at least one promoter on a support material and the promoter can be selected from the group of Co, Mn, Ti, Zr, V, Nb, K, Cs, Ga, B, P, Rb, Ag, Na, Cu, Mg, Fe, Mo, Ce, and combinations thereof.

An embodiment of the present invention, either by itself or in combination with other embodiments, is a process for making styrene that includes reacting toluene with a C₁ source over a catalyst in a first reactor producing a first product stream comprising a gas fraction and a liquid fraction. The first product stream is separated in one or more separation units, and a liquid fraction including styrene and ethylbenzene is fed to a second reactor and a gas fraction that can include carbon monoxide and hydrogen is fed to a third reactor. The carbon monoxide and hydrogen are reacted with oxygen in the third reactor, making a third product stream of carbon dioxide and steam. The third product stream is fed into the second reactor, where styrene and ethylbenzene are produced in a second product stream. The first reactor can be an alkylation reactor, the third reactor can be an oxidation reactor, and the second reactor can be an oxy-dehydrogenation reactor, where ethylbenzene is dehydrogenated to form styrene.

An embodiment of the present invention, either by itself or in combination with other embodiments, is a process for making styrene that includes reacting toluene with a C₁ source over a catalyst in a first reactor producing a first product stream of styrene and ethylbenzene, and feeding (i) the first product stream, wherein the first product stream has a first molar ratio of styrene to ethylbenzene, and (ii) an oxidant, into a second reactor to form a second product stream of styrene and ethylbenzene, where the second product stream has a second molar ratio of styrene to ethylbenzene and the second molar ratio is greater than the first molar ratio. The oxidant can be carbon dioxide.

The various aspects of the present invention can be joined in combination with other aspects of the invention and the listed embodiments herein are not meant to limit the invention. All combinations of aspects of the invention are enabled, even if not given in a particular example herein.

### BRIEF DESCRIPTION OF DRAWINGS

FIG. 1 illustrates a simplified flow chart for the production of styrene by the reaction of formaldehyde and toluene.

FIG. 2 illustrates another simplified flow chart for the production of styrene by the reaction of formaldehyde and toluene.

### DETAILED DESCRIPTION

In an embodiment, toluene is reacted with a carbon source, which can be referred to as a C₁ source, to produce a product stream having a liquid fraction that can include styrene, toluene, ethylbenzene, and combinations thereof, and a gas fraction including hydrogen and carbon monoxide. The styrene, unreacted toluene, and ethylbenzene can be further separated from the product stream and fed into a reactor in the presence of a co-feed formed from the reaction of oxygen with a mixture of carbon monoxide and hydrogen separated from the product stream, thereby producing unreacted toluene, styrene and ethylbenzene, wherein the molar ratio of styrene to ethylbenzene is greater than the molar ratio of styrene to ethylbenzene produced in the original reaction of toluene with the C₁ source. In an embodiment, the C₁ source includes methanol or formaldehyde or a mixture of the two. In an embodiment, the co-feed includes carbon dioxide (CO₂). In an embodiment, the co-feed including carbon dioxide is fed into an oxy-dehydrogenation reactor, wherein the carbon dioxide drives the oxy-dehydrogenation of ethylbenzene. In an alternative embodiment, toluene is reacted with one or more of the following: formalin, trioxane, methylformcel, paraformaldehyde, dimethyl ether, and methylal. In a further embodiment, the C₁ source is selected from the group of methanol, formaldehyde, formalin (37-50% H₂CO in solution of water and methanol), trioxane (1,3,5-trioxane), methylformcel (55% H₂CO in methanol), paraformaldehyde, dimethyl ether, methylal (dimethoxymethane), and combinations thereof.

Formaldehyde can be produced either by the oxidation or dehydrogenation of methanol.

In an embodiment, formaldehyde is produced by the dehydrogenation of methanol to produce formaldehyde and hydrogen gas. This reaction step produces a dry formaldehyde stream that may be preferred, as it would not require the separation of the water prior to the reaction of the formaldehyde with toluene. The dehydrogenation process is described in the equation below:

CH₃OH→CH₂O+H₂

Formaldehyde can also be produced by the oxidation of methanol to produce formaldehyde and water. The oxidation of methanol is described in the equation below:

2CH₃OH+O₂→2CH₂O+2H₂O

In the case of using a separate process to obtain formaldehyde, a separation unit may then be used in order to separate the formaldehyde from the hydrogen gas or water from the formaldehyde and unreacted methanol prior to reacting the formaldehyde with toluene for the production of styrene. This separation would inhibit the hydrogen from entering the alkylation reactor, thus limiting the hydrogenation of styrene to ethylbenzene. Purified formaldehyde could then be sent to an alkylation reactor and the unreacted methanol could be recycled.

Although the reaction has a 1:1 molar ratio of toluene and the C₁ source, the ratio of the C₁ source and toluene feedstreams is not limited within the present invention and can vary depending on operating conditions and the efficiency of the reaction system. If excess toluene or C₁ source is fed to the reaction zone, the unreacted portion can be subsequently separated and recycled back into the process. In one embodiment the ratio of toluene:C₁ source can range from between 100:1 to 1:100. In alternate embodiments the ratio of toluene:C₁ source can range from 50:1 to 1:50; from 20:1 to 1:20; from 10:1 to 1:10; from 5:1 to 1:5; from 2:1 to 1:2.

In an embodiment, certain products of the reaction between toluene and the C₁ source, including styrene, unreacted toluene, and ethylbenzene, are further combined with a co-feed in a reactor. In an embodiment, the reactor is an oxy-dehydrogenation reactor and the co-feed includes carbon dioxide. In another embodiment, the co-feed may be combined with nitrogen prior to combining the co-feed with the products. The co-feed may be combined with the products in any desired amounts. In an embodiment the co-feed can be added to the ethylbenzene from the reaction between toluene and the C₁ source in a EB:CO₂ molar ratio of at least 5:1. In another embodiment the co-feed can be added to the ethylbenzene from the reaction between toluene and the C₁ source in a EB:CO₂ molar ratio ranging from 10:1 to 1:1.

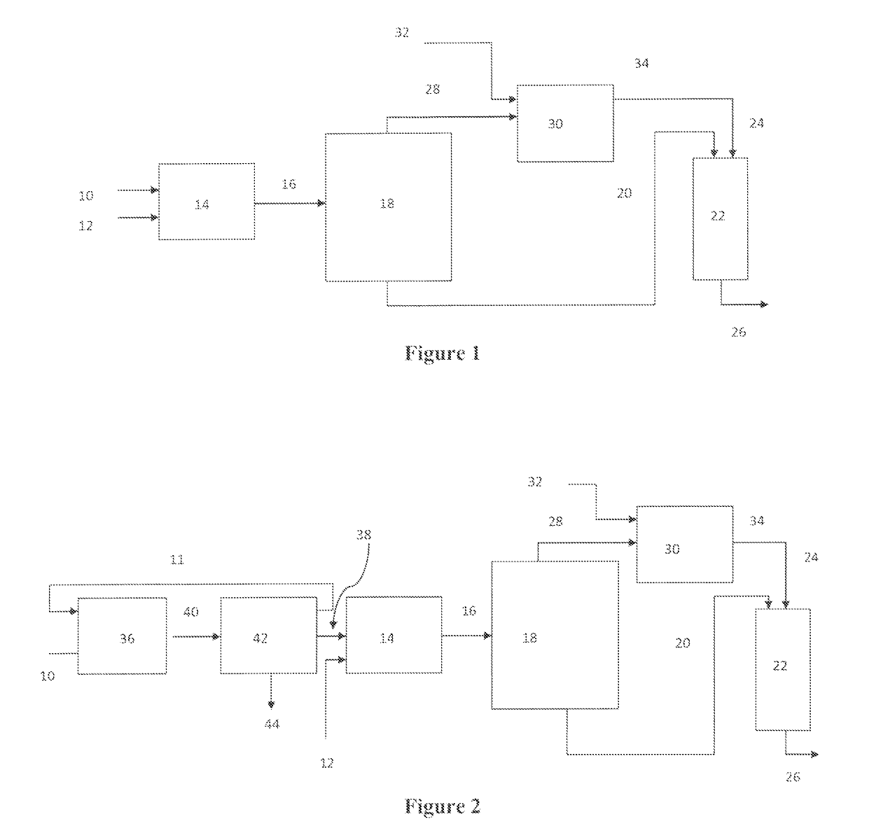

Turning now to the Figures, FIG. 1 illustrates a simplified flow chart of one embodiment of the styrene production process described above. A feed stream containing a C₁ source (10) including methanol is fed along with a feed stream of toluene (12) into a first reactor (14). In the embodiment shown in FIG. 1, the first reactor (14) is an alkylation reactor. The methanol (10) reacts with a catalyst in the alkylation reactor (14) to produce formaldehyde. The toluene (12) and formaldehyde react to produce a first product stream (16), namely an alkylation product stream, which can include a liquid fraction including styrene, ethylbenzene, and unreacted toluene, and a gas fraction including carbon monoxide and hydrogen. The alkylation product stream (16) is sent to a first separation unit (18). In an embodiment, the first separation unit (18) includes one or more distillation units where the gas fractions and liquid fractions of the alkylation product stream (16) may be separated and routed as shown in FIG. 1.

The alkylation product stream further has a first molar ratio of styrene to ethylbenzene. In an embodiment, the first molar ratio of styrene to ethylbenzene is less than one. In an alternate embodiment, the first molar ratio of styrene to ethylbenzene is equal to one. In an alternate embodiment, the first molar ratio of styrene to ethylbenzene is greater than one.

The styrene, ethylbenzene, and unreacted toluene (collectively labeled as (20)) are separated from the alkylation product stream (16) in one or more distillation units (18) as shown in FIG. 1 and fed into a second reactor (22) with a co-feed (24) including carbon dioxide and steam, which will be discussed further below. In the embodiment of FIG. 1, the second reactor is an oxy-dehydrogenation reactor (22) and the reaction in the oxy-dehydrogenation reactor produces a second product stream (26), namely an oxy-dehydrogenation product stream, further discussed below. Optionally, any unreacted toluene from the distillation unit (18) or from the second product stream (26) after further separation (not shown) may be fed back into the alkylation reactor (14). Optionally, the styrene may be separated in the distillation unit (18) and fed into the oxy-dehydrogenation product stream (26).

As illustrated in FIG. 1, a mixture (28) primarily composed of carbon monoxide and hydrogen is separated from the alkylation product stream (16) in the distillation unit (18) and fed to a third reactor (30), wherein the third reactor is an oxidation reactor in the embodiment shown. The mixture (28) is fed into the oxidation reactor (30) in the presence of a co-feed (32) including oxygen, wherein a reaction occurs and carbon dioxide and steam are produced in a third product stream (34) formed from the reaction. In the embodiment of FIG. 1, the third product stream (34) is an oxidation product stream. The resulting co-feed (24) from the oxidation product stream (34), namely carbon dioxide and steam, is fed into the oxy-dehydrogenation reactor (22) with the styrene, ethylbenzene, and unreacted toluene (20) separated from the alkylation product stream (16), thereby forming an oxy-dehydrogenation product stream (26) including styrene, ethylbenzene, and unreacted toluene.

The oxy-dehydrogenation product stream (26) further has a second molar ratio of styrene to ethylbenzene, wherein the second molar ratio is greater than the first molar ratio of styrene to ethylbenzene formed from the reaction of toluene with the C₁ source in stream (20). The oxy-dehydrogenation product stream (26) can be subjected to further treatment or processing if desired. Optionally, the oxy-dehydrogenation product stream (26) may be fed back to the distillation unit (18) for further processing. Optionally, the oxy-dehydrogenation product stream may be fed back to the oxy-dehydrogenation reactor for further processing.

The operating conditions of the reactors and separators will be system specific and can vary depending on the feedstream composition and the composition of the product streams. The alkylation reactor for the reaction of a C₁ source including methanol to formaldehyde and the reaction of toluene with formaldehyde will operate at elevated temperatures and pressures and may contain a basic or neutral catalyst system. The temperature can range in a non-limiting example from 250° C. to 750° C., optionally from 300° C. to 500° C., optionally from 375° C. to 450° C. The pressure can range in a non-limiting example from 0.1 atm to 70 atm, optionally from 0.1 atm to 35 atm, optionally from 0.1 atm to 10 atm, optionally from 0.1 atm to 5 atm.

Looking now at FIG. 2, a simplified flow chart is shown of an alternate embodiment of the styrene process discussed in FIG. 1. In this embodiment, a preliminary reactor (36) is introduced as an initial stage in the styrene production process and is designed to convert the methanol feed (10) into formaldehyde (38). The preliminary reactor is either a dehydrogenation reactor or an oxidation reactor. A preliminary product stream (40) is formed from the reaction of the methanol feed (10) in the preliminary reactor (36) and the preliminary product stream is sent to a preliminary separation unit (42) where the formaldehyde (38) is separated from any unreacted methanol (11) and unwanted byproducts (44). Any unreacted methanol (11) can be recycled back into the preliminary reactor (36). Any unwanted byproducts (44) can be separated from the formaldehyde (38).

In one embodiment, the preliminary reactor (36) is a dehydrogenation reactor that produces a preliminary product stream including formaldehyde and hydrogen and the preliminary separation unit is a membrane capable of removing hydrogen from the preliminary product stream.

In an alternate embodiment the preliminary reactor (36) is an oxidation reactor that produces a preliminary product stream including formaldehyde and water. The preliminary product stream including formaldehyde and water can then be sent to the alkylation reactor without being routed through the preliminary separation unit.

As shown in FIG. 2, the formaldehyde feed stream (38) and a feed stream of toluene (12) are fed into a first reactor (14). The first reactor (14) is an alkylation reactor in the embodiment illustrated in FIG. 2. The toluene (12) and formaldehyde (38) react to produce a first product stream (16), namely an alkylation product stream, which can include a liquid fraction that can include styrene, toluene, ethylbenzene, and combinations thereof, and a gas fraction that can include carbon monoxide and hydrogen. In an embodiment, the toluene and formaldehyde react over a catalyst in the alkylation reactor (14). The alkylation product stream (16) has a first molar ratio of styrene to ethylbenzene. In an embodiment, the first molar ratio of styrene to ethylbenzene is less than one. In an alternate embodiment, the first molar ratio of styrene to ethylbenzene is equal to one. In an alternate embodiment, the first molar ratio of styrene to ethylbenzene is greater than one. The alkylation product stream (16) is sent to one or more distillation units (18). The gas fraction and liquid fraction of the alkylation product stream (16) can be separated and routed in the same manner as shown in FIG. 1 and as discussed above.

The operating conditions of the reactors and separators will be system specific and can vary depending on the feedstream composition and the composition of the product streams. The alkylation reactor for the reaction of toluene and formaldehyde will operate at elevated temperatures and pressures and may contain a basic or neutral catalyst system. The temperature can range in a non-limiting example from 250° C. to 750° C., optionally from 300° C. to 500° C., optionally from 375° C. to 450° C. The pressure can range in a non-limiting example from 0.1 atm to 70 atm, optionally from 0.1 atm to 35 atm, optionally from 0.1 atm to 10 atm, optionally from 0.1 atm to 5 atm.

Improvement in side chain alkylation selectivity may be achieved by treating a molecular sieve zeolite catalyst with chemical compounds to inhibit the external acidic sites and minimize aromatic alkylation on the ring positions. Another means of improvement of side chain alkylation selectivity can be to inhibit overly basic sites, such as for example with the addition of a boron compound. Another means of improvement of side chain alkylation selectivity can be to impose restrictions on the catalyst structure to facilitate side chain alkylation. In one embodiment the catalyst used in an embodiment of the present invention is a basic or neutral catalyst.

The catalytic reaction systems suitable for this invention can include one or more of the zeolite, crystalline or amorphous materials modified for side chain alkylation selectivity. A non-limiting example can be a zeolite promoted with one or more of the following: Co, Mn, Ti, Zr, V, Nb, K, Cs, Ga, B, P, Rb, Ag, Na, Cu, Mg, Fe, Mo, Ce, or combinations thereof. In an embodiment, the zeolite can be promoted with one or more of Ce, Cu, P, Cs, B, Co, Ga, or combinations thereof. The promoter can exchange with an element within the zeolite or amorphous material and/or be attached to the zeolite or amorphous material in an occluded manner.

In an embodiment, the catalyst contains greater than 0.1 wt % of at least one promoter based on the total weight of the catalyst. In another embodiment, the catalyst contains up to 5 wt % of at least one promoter. In a further embodiment, the catalyst contains from 0.1 to 3 wt % of at least one promoter. In an embodiment, the at least one promoter is boron.

Zeolite materials suitable for this invention may include silicate-based zeolites and amorphous compounds such as faujasite, mordenite, chabazite, offretite, clinoptilolite, erionite, sihealite, and the like. Silicate-based zeolites are made of alternating SiO₄ and MO_{X} tetrahedra, where M is an element selected from the Groups 1 through 16 of the Periodic Table (new IUPAC). These types of zeolites have 4-, 6-, 8-, 10-, or 12-membered oxygen ring channels. An example of zeolites of this invention can include faujasites. Other suitable zeolite materials include zeolite A, zeolite L, zeolite beta, zeolite X, zeolite Y, ZSM-5, MCM-22, and MCM-41. In a more specific embodiment, the zeolite is an X-type zeolite.

In an embodiment, the zeolite materials suitable for this invention are characterized by silica to alumina ratio (Si/Al) of less than 1.5. In another embodiment, the zeolite materials are characterized by a Si/Al ratio ranging from 1.0 to 200, optionally from 1.0 to 100, optionally from 1.0 to 50, optionally from 1.0 to 10, optionally from 1.0 to 2.0, optionally from 1.0 to 1.5.

The various elements that make up the catalyst can be derived from any suitable source, such as in their elemental form, or in compounds or coordination complexes of an organic or inorganic nature, such as carbonates, oxides, hydroxides, nitrates, acetates, chlorides, phosphates, sulfides and sulfonates. The elements and/or compounds can be prepared by any suitable method, known in the art, for the preparation of such materials.

The term “substrate” as used herein is not meant to indicate that this component is necessarily inactive, while the other metals and/or promoters are the active species. On the contrary, the substrate can be an active part of the catalyst. The term “substrate” would merely imply that the substrate makes up a significant quantity, generally 10% or more by weight, of the entire catalyst. The promoters individually can range from 0.01% to 60% by weight of the catalyst, optionally from 0.01% to 50. If more than one promoter is combined, they together generally can range from 0.01% up to 70% by weight of the catalyst. The elements of the catalyst composition can be provided from any suitable source, such as in its elemental form, as a salt, as a coordination compound, etc.

The addition of a support material to improve the catalyst physical properties is possible within the present invention. Binder material, extrusion aids or other additives can be added to the catalyst composition or the final catalyst composition can be added to a structured material that provides a support structure. For example, the final catalyst composition can include an alumina or aluminate framework as a support. Upon calcination these elements can be altered, such as through oxidation which would increase the relative content of oxygen within the final catalyst structure. The combination of the catalyst of the present invention combined with additional elements such as a binder, extrusion aid, structured material, or other additives, and their respective calcination products, are included within the scope of the invention.

In one embodiment, the catalyst can be prepared by combining a substrate with at least one promoter element. Embodiments of a substrate can be a molecular sieve, from either natural or synthetic sources. Zeolites and zeolite-like materials can be an effective substrate. Alternate molecular sieves also contemplated are zeolite-like materials such as the crystalline silicoaluminophosphates (SAPO) and the aluminophosphates (ALPO).

The present invention is not limited by the method of catalyst preparation, and all suitable methods should be considered to fall within the scope herein. Particularly effective techniques are those utilized for the preparation of solid catalysts. Conventional methods include co-precipitation from an aqueous, an organic or a combination solution-dispersion, impregnation, dry mixing, wet mixing or the like, alone or in various combinations. In general, any method can be used which provides compositions of matter containing the prescribed components in effective amounts. According to an embodiment the substrate is charged with promoter via an incipient wetness impregnation. Other impregnation techniques such as by soaking, pore volume impregnation, or percolation can optionally be used. Alternate methods such as ion exchange, wash coat, precipitation, and gel formation can also be used. Various methods and procedures for catalyst preparation are listed in the technical report Manual of Methods and Procedures for Catalyst Characterization by J. Haber, J. H. Block and B. Dolmon, published in the International Union of Pure and Applied Chemistry, Volume 67, Nos. 8/9, pp. 1257-1306, 1995, incorporated herein in its entirety.

In an aspect, the at least one promoter includes boron. In an embodiment, the catalyst contains greater than 0.1 wt % boron based on the total weight of the catalyst. In another embodiment, the catalyst contains from 0.1 to 3 wt % boron, optionally from 0.1 to 1 wt % boron.

The boron promoter can be added to the catalyst by contacting the substrate, impregnation, or any other method, with any known boron source. In an embodiment, the boron source is selected from the group of boric acid, boron phosphate, methoxyboroxine, methylboroxine, and trimethoxyboroxine and combinations thereof. In another embodiment, the boron source contains boroxines. In a further embodiment, the boron source is selected from the group of methoxyboroxine, methylboroxine, and trimethoxyboroxine and combinations thereof.

In an embodiment, a substrate may be previously treated with a boron source prior to an addition of at least one promoter, wherein the at least one promoter includes boron. In another embodiment, a boron treated zeolite may be combined with at least one promoter, wherein the at least one promoter includes boron. In a further embodiment, boron may be added to the catalyst system by adding at least one promoter containing boron as a co-feed with toluene and methanol. In an even further embodiment, boron may be added to the catalyst system by adding boroxines as a co-feed with toluene and methanol. The boroxines can include, methoxyboroxine, methylboroxine, and trimethoxyboroxine, and combinations thereof. The boron treated zeolite further combined with at least one promoter including boron may be used in preparing a supported catalyst such as extrudates and tablets.

The prepared catalyst can be ground, pressed, sieved, shaped and/or otherwise processed into a form suitable for loading into a reactor. The reactor can be any type known in the art to make catalyst particles, such as a fixed bed, fluidized bed, or swing bed reactor. Optionally an inert material can be used to support the catalyst bed and to place the catalyst within the bed.

Embodiments of reactors that can be used with the present invention can include, by non-limiting examples: fixed bed reactors; fluid bed reactors; and entrained bed reactors. Reactors capable of the elevated temperature and pressure as described herein, and capable of enabling contact of the reactants with the catalyst, can be considered within the scope of the present invention. Embodiments of the particular reactor system may be determined based on the particular design conditions and throughput, as by one of ordinary skill in the art, and are not meant to be limiting on the scope of the present invention. An example of a suitable reactor can be a fluid bed reactor having catalyst regeneration capabilities. This type of reactor system employing a riser can be modified as needed, for example by insulating or heating the riser if thermal input is needed, or by jacketing the riser with cooling water if thermal dissipation is required. These designs can also be used to replace catalyst while the process is in operation, by withdrawing catalyst from the regeneration vessel from an exit line or adding new catalyst into the system while in operation.

In another aspect, the one or more reactors may include one or more catalyst beds. In the event of multiple beds, an inert material layer can separate each bed. The inert material can comprise any type of inert substance. In an embodiment, a reactor includes between 1 and 25 catalyst beds. In a further embodiment, a reactor includes between 2 and 10 catalyst beds. In a further embodiment, a reactor includes between 2 and 5 catalyst beds. In addition, the C₁ source and/or toluene may be injected into a catalyst bed, an inert material layer, or both. In a further embodiment, at least a portion of the C₁ source is injected into a catalyst bed(s) and at least a portion of the toluene feed is injected into an inert material layer(s).

In an alternate embodiment, the entire C₁ source is injected into a catalyst bed(s) and all of the toluene feed is injected into an inert material layer(s). In another aspect, at least a portion of the toluene feed is injected into a catalyst bed(s) and at least a portion the C₁ source is injected into an inert material layer(s). In a further aspect, all of the toluene feed is injected into a catalyst bed(s) and the entire C₁ source is injected into an inert material layer(s).

The toluene and C₁ source coupling reaction may have a toluene conversion percent greater than 0.01 mol %. In an embodiment the toluene and C₁ source coupling reaction is capable of having a toluene conversion percent in the range of from 0.05 mol % to 40 mol %. In a further embodiment the toluene and C₁ source coupling reaction is capable of having a toluene conversion in the range of from 2 mol % to 40 mol %, optionally from 5 mol % to 35 mol %, optionally from 10 mol % to 25 mol %.

In an aspect the toluene and C₁ source coupling reaction is capable of selectivity to styrene greater than 1 mol %. In another aspect, the toluene and C₁ source coupling reaction is capable of selectivity to styrene in the range of from 1 mol % to 99 mol %. In an aspect the toluene to a C₁ source coupling reaction is capable of selectivity to ethylbenzene greater than 1 mol %. In another aspect, the toluene and C₁ source coupling reaction is capable of selectivity to ethylbenzene in the range of from 1 mol % to 99 mol %. In an aspect the toluene and C₁ source coupling reaction is capable of yielding less than 0.5 mol % of ring alkylated products such as xylenes.

The term “conversion” refers to the percentage of reactant (e.g. toluene) that undergoes a chemical reaction.

X_{Tol}=conversion of toluene (mol %)=(Tolᵢₙ−Tolₒᵤₜ)/Tolᵢₙ

X_{MeOH}=conversion of methanol to styrene+ethylbenzene (mol %)

The term “molecular sieve” refers to a material having a fixed, open-network structure, usually crystalline, that may be used to separate hydrocarbons or other mixtures by selective occlusion of one or more of the constituents, or may be used as a catalyst in a catalytic conversion process.

Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

The term “regenerated catalyst” refers to a catalyst that has regained enough activity to be efficient in a specified process. Such efficiency is determined by individual process parameters.

The term “selectivity” refers to the relative activity of a catalyst in reference to a particular compound in a mixture. Selectivity is quantified as the proportion of a particular product relative to all other products.

S_{Sty}=selectivity of toluene to styrene (mol %)=Styₒᵤₜ/Tol_{converted}×100

S_{Bz}=selectivity of toluene to benzene (mol %)=Benzeneₒᵤₜ/Tol_{converted}×100

S_{EB}=selectivity of toluene to ethylbenzene (mol %)=EBₒᵤₜ/Tol_{converted}×100

S_{Xy1}=selectivity of toluene to xylenes (mol %)=Xylenesₒᵤₜ/Tol_{converted}×100

S_{sty+EB} (MeOH)=selectivity of methanol to styrene+ethylbenzene (mol %)=(Styₒᵤₜ+EBₒᵤₜ)/MeOH_{converted}×100

The term “spent catalyst” refers to a catalyst that has lost enough catalyst activity to no longer be efficient in a specified process. Such efficiency is determined by individual process parameters.

The term “zeolite” refers to a molecular sieve containing an aluminosilicate lattice, usually in association with some aluminum, boron, gallium, iron, and/or titanium, for example. In the following discussion and throughout this disclosure, the terms molecular sieve and zeolite will be used more or less interchangeably. One skilled in the art will recognize that the teachings relating to zeolites are also applicable to the more general class of materials called molecular sieves.

The term “liquid fraction” refers to the one or more components of a total number of components in a product stream being in a liquid phase.

The term “gas fraction” refers to the one or more components of a total number of components in a product stream being in a gas phase.

The various aspects of the present invention can be joined in combination with other aspects of the invention and the listed embodiments herein are not meant to limit the invention. All combinations of various aspects of the invention are enabled, even if not given in a particular example herein.

While illustrative embodiments have been depicted and described, modifications thereof can be made by one skilled in the art without departing from the spirit and scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.).

Depending on the context, all references herein to the “invention” may in some cases refer to certain specific embodiments only. In other cases it may refer to subject matter recited in one or more, but not necessarily all, of the claims. While the foregoing is directed to embodiments, versions and examples of the present invention, which are included to enable a person of ordinary skill in the art to make and use the inventions when the information in this patent is combined with available information and technology, the inventions are not limited to only these particular embodiments, versions and examples. Also, it is within the scope of this disclosure that the aspects and embodiments disclosed herein are usable and combinable with every other embodiment and/or aspect disclosed herein, and consequently, this disclosure is enabling for any and all combinations of the embodiments and/or aspects disclosed herein. Other and further embodiments, versions and examples of the invention may be devised without departing from the basic scope thereof and the scope thereof is determined by the claims that follow.

What is claimed is:

1. A process for making styrene comprising:
reacting toluene with a C₁ source in the presence of a catalyst in a first reactor to form a first product stream;
separating the first product stream into a gas fraction comprising carbon monoxide and a liquid fraction comprising ethylbenzene;
reacting the gas fraction with oxygen to form a co-feed that comprises carbon dioxide;
feeding at least a portion of the liquid fraction into a second reactor with the co-feed to form a second product stream comprising styrene.

2. The process of claim 1, wherein the first reactor is an alkylation reactor.

3. The process of claim 1, wherein the second reactor is an oxy-dehydrogenation reactor.

4. The process of claim 1, wherein the first product stream comprises styrene, ethylbenzene, carbon monoxide and hydrogen.

5. The process of claim 1, wherein the gas fraction comprises carbon monoxide and hydrogen.

6. The process of claim 1, wherein the liquid fraction comprises styrene, ethylbenzene, and unreacted toluene.

7. The process of claim 3, wherein a portion of the ethylbenzene from the first product stream is dehydrogenated to styrene by reaction with the carbon dioxide in the co-feed within the oxy-dehydrogenation reactor.

8. The process of claim 1, wherein the molar ratio of styrene to ethylbenzene in the second product stream is greater than the molar ratio of styrene to ethylbenzene in the first product stream.

9. The process of claim 1, wherein the C₁ source is selected from the group consisting of methanol, formaldehyde, formalin, trioxane, methylformcel, paraformaldehyde, methylal, dimethyl ether, and combinations thereof.

9. The process of claim 1, wherein the catalyst comprises at least one promoter on a support material.

10. The process of claim 9, wherein the promoter is selected from the group consisting of Co, Mn, Ti, Zr, V, Nb, K, Cs, Ga, B, P, Rb, Ag, Na, Cu, Mg, Fe, Mo, Ce, and combinations thereof.

11. A process for making styrene comprising:
reacting toluene with a C₁ source over a catalyst in a first reactor producing a first product stream comprising a gas fraction and a liquid fraction;
separating the first product stream in one or more separation units, wherein the liquid fraction comprising styrene and ethylbenzene is fed to a second reactor and the gas fraction comprising carbon monoxide and hydrogen is fed to a third reactor;
reacting the carbon monoxide and hydrogen with oxygen in the third reactor, wherein a third product stream comprising carbon dioxide and steam is produced; and
feeding the third product stream into the second reactor, wherein styrene and ethylbenzene are produced in a second product stream.

12. The process of claim 11, wherein the first reactor is an alkylation reactor, the third reactor is an oxidation reactor, and the second reactor is an oxy-dehydrogenation reactor, wherein ethylbenzene is dehydrogenated to form styrene in the second reactor.

13. The process of claim 11, wherein the molar ratio of styrene to ethylbenzene in the second product stream is greater than the molar ratio of styrene to ethylbenzene in the first product stream.

14. The process of claim 11, wherein the C₁ source is selected from the group consisting of methanol, formaldehyde, formalin, trioxane, methylformcel, paraformaldehyde, methylal, dimethyl ether, and combinations thereof.

15. The process of claim 11, wherein the catalyst comprises at least one promoter on a support material, wherein the promoter is selected from the group consisting of Co, Mn, Ti, Zr, V, Nb, K, Cs, Ga, B, P, Rb, Ag, Na, Cu, Mg, Fe, Mo, Ce, and combinations thereof.

16. A process for making styrene comprising:
reacting toluene with a C₁ source over a catalyst in a first reactor producing a first product stream comprising styrene and ethylbenzene; and
feeding (i) the first product stream, wherein the first product stream has a first molar ratio of styrene to ethylbenzene, and (ii) an oxidant, into a second reactor to form a second product stream comprising styrene and ethylbenzene, wherein the second product stream has a second molar ratio of styrene to ethylbenzene and the second molar ratio is greater than the first molar ratio.

17. The process of claim 16, wherein the oxidant is carbon dioxide.

18. The process of claim 16, further including oxidizing a mixture separated from the first product stream to form the oxidant, wherein the mixture comprises carbon monoxide and hydrogen and the oxidant is carbon dioxide.

19. The process of claim 16, wherein the C₁ source is selected from the group consisting of methanol, formaldehyde, formalin, trioxane, methylformcel, paraformaldehyde, methylal, dimethyl ether, and combinations thereof.

20. The process of claim 16, wherein the catalyst comprises at least one promoter on a support material, wherein the promoter is selected from the group consisting of Co, Mn, Ti, Zr, V, Nb, K, Cs, Ga, B, P, Rb, Ag, Na, Cu, Mg, Fe, Mo, Ce, and combinations thereof.
